(19) United States
(12) Patent Application Publication
(10) Pub. No.: US 2024/0264528 A1
(43) Pub. Date: Aug. 8, 2024

(54) PHOTORESIST UNDERLAYER COMPOSITIONS AND METHODS OF FORMING ELECTRONIC DEVICES

(71) Applicant: ROHM AND HAAS ELECTRONIC MATERIALS LLC, Marlborough, MA (US)

(72) Inventors: Sheng LIU, Bow, NH (US); Iou-Sheng KE, Andover, MA (US); Keren ZHANG, Kirkland, WA (US); James F. CAMERON, Brookline, MA (US); Shintaro YAMADA, Shrewsbury, MA (US); Li CUI, Westborough, MA (US)

(21) Appl. No.: 18/537,020

(22) Filed: Dec. 12, 2023

Related U.S. Application Data
(60) Provisional application No. 63/436,573, filed on Dec. 31, 2022.

(51) Int. Cl. G03F 7/11; C07C 43/285; C07C 49/255; C07C 317/22

(52) U.S. Cl. CPC G03F 7/11; C07C 43/285; C07C 49/255; C07C 317/22

(57) ABSTRACT

Photoresist underlayer compositions, comprising: a curable compound comprising a group of the following formula (1):

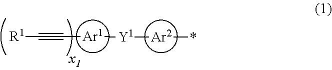

wherein: R¹ is each independently H, C₁₋₃₀ alkyl, or C₃₋₃₀ cycloalkyl; Ar¹ is an aromatic ring or a fused aromatic ring system having from 5 to 30 carbon atoms, wherein Ar¹ is substituted or unsubstituted; Ar² is an aromatic ring chosen from a 6-membered carbocyclic aromatic ring, a 5- or 6-membered heteroaromatic ring, or a fused aromatic ring system having from 5 to 30 carbon atoms, wherein Ar² optionally comprises a fused cyclic imide moiety, a fused oxazole moiety, a fused imidazole moiety, or a fused thiazole moiety, and wherein Ar² is substituted or unsubstituted; Y¹ is a single covalent bond, or is selected from —O—, —C(O)—, —C(O)O—, —S—, —S(O)₂—, —N(R²)—, —C(O)N(R²)—, —C(O)N(R²)C(O)—, —(CH₂)_{y}—, or a combination thereof, wherein R² is H, C₁₋₁₀ alkyl, C₂₋₁₀ unsaturated hydrocarbyl, C₅₋₃₀ aryl, C(O)R³, or S(O)₂R³, wherein R³ is chosen from H, C₁₋₁₀ alkyl, C₂₋₁₀ unsaturated hydrocarbyl, and C₅₋₃₀ aryl, and y is an integer from 1 to 6; x is an integer from 2 to 5; and * denotes a binding site to a part of the curable compound other than the group represented by formula (1), provided that no two

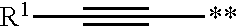

groups are in an ortho position to each other on Ar¹, wherein ** denotes the point of attachment to an aromatic ring carbon of Ar¹; and
a solvent.

### BACKGROUND OF THE INVENTION

The present invention relates generally to the field of electronic device manufacture, and more specifically to the field of photoresist underlayer compositions for use in the manufacture of electronic devices such as semiconductor devices.

It is well-known in lithographic processes that a resist pattern can collapse due to surface tension from the developer used if the resist pattern is relatively tall versus its width (high aspect ratio). Multilayer resist processes (such as three- and four-layer processes) have been devised which can address this issue of pattern collapse where a high aspect ratio is desired. Such multilayer processes use a resist top layer, one or more middle layers, and a bottom layer (or underlayer). In such multilayer resist processes, the top photoresist layer is imaged and developed in typical fashion to provide a resist pattern. The pattern is then transferred to the one or more middle layers, typically by etching. Each middle layer is selected such that a different etch process is used, for example, different plasma etches. Finally, the pattern is transferred to the underlayer, typically by etching. Such middle layers may be composed of various materials and typically have antireflective properties, while the underlayer materials are typically composed of high carbon content materials. The underlayer material is typically selected to provide desired antireflective properties, planarizing properties, as well as etch selectivity.

The incumbent technologies for underlayer formation include chemical vapor deposited (CVD) carbon and solution-processed high-carbon content polymers. The CVD materials have several significant limitations including high cost of ownership, inability to form a planarizing layer over topography on a substrate, and high absorbance at 633 nm which is used for pattern alignment. For these reasons, the industry has been moving to solution-processed high-carbon content materials for underlayers. It is typically desired that the underlayer materials have the following properties: capable of being cast onto a substrate by spin-coating to form a uniform coating; thermal-set upon heating with low out-gassing and sublimation; soluble in common processing solvents for good equipment compatibility; have appropriate optical properties (e.g., n and k values) to work in conjunction with currently used silicon hardmask and bottom antireflectant (BARC) layers to impart low reflectivity necessary for photoresist imaging; high carbon content for reduced etching rate; low- to medium-temperature curing; thermally stable up to >400° C. so as to not be damaged during subsequent processes; good gap-fill and planarization properties; and resistant to stripping by common solvents used in overcoated photoresist or other layers.

It is well-known that materials of relatively low molecular weight have relatively low viscosity, and can flow into features in a substrate, such as vias and trenches, to afford planarizing layers. Underlayer materials should be able to planarize with relatively low out-gassing up to 400° C. For use as a high-carbon content underlayer, it is desired for any composition to be thermally set upon heating.

US2020/0142309A1 discloses aromatic photoresist underlayer compositions comprising one or more curable compounds comprising an aromatic core chosen from a C₅₋₆ aromatic ring and a C₉₋₃₀ fused aromatic ring system and three or more substituents of formula (1):

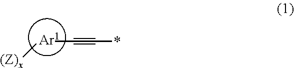

wherein at least two substituents of formula (1) are attached to the aromatic core; and wherein Ar¹ is an aromatic ring or fused aromatic ring system having from 5 to 30 carbons; Z is a substituent chosen from OR¹, protected hydroxyl, carboxyl, protected carboxyl, SR¹, protected thiol, —O—C(═O)—C₁₋₆-alkyl, halogen, and NHR²; each R¹ is chosen from H, C₁₋₁₀ alkyl, C₂₋₁₀ unsaturated hydrocarbyl, and C₅₋₃₀ aryl; each R² is chosen from H, C₁₋₁₀ alkyl, C₂₋₁₀ unsaturated hydrocarbyl, C₅₋₃₀ aryl, C(═O)—R¹, and S(═O)₂—R¹; x is an integer from 1 to 4; and * denotes the point of attachment to the aromatic core. While the described photoresist underlayer compositions have beneficial properties, the need for compositions allowing for increased thermal stability at higher cure temperatures is becoming of increased importance.

There is a need in the art for photoresist underlayer compositions useful in forming electronic devices, and for methods of using such compositions, that that address one or more problems associated with the state of the art.

### SUMMARY OF THE INVENTION

In accordance with a first aspect of the invention, underlayer compositions are provided. The underlayer compositions comprise: a curable compound comprising a group of the following formula (1):

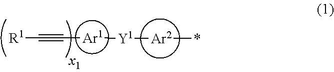

wherein: R¹ is each independently H, C₁₋₃₀ alkyl, or C₃₋₃₀ cycloalkyl; Ar¹ is an aromatic ring or a fused aromatic ring system having from 5 to 30 carbon atoms, wherein Ar¹ is substituted or unsubstituted; Ar² is an aromatic ring chosen from a 6-membered carbocyclic aromatic ring, a 5- or 6-membered heteroaromatic ring, or a fused aromatic ring system having from 5 to 30 carbon atoms, wherein Ar² optionally comprises a fused cyclic imide moiety, a fused oxazole moiety, a fused imidazole moiety, or a fused thiazole moiety, and wherein Ar² is substituted or unsubstituted; Y¹ is a single covalent bond, or is selected from —O—, —C(O)—, —C(O)O—, —S—, —S(O)₂—, —N(R²)—, —C(O)N(R²)—, —C(O)N(R²)C(O)—, —(CH²)_{y}—, or a combination thereof, wherein R² is H, C₁₋₁₀-alkyl, C₂₋₁₀-unsaturated hydrocarbyl, C₅₋₃₀-aryl, C(O)—R³, or S(O)₂—R³, wherein R³ is chosen from H, C₁₋₁₀-alkyl, C₂₋₁₀-unsaturated hydrocarbyl, and C₅₋₃₀-aryl, and y is an integer from 1 to 6; x₁ is an integer from 2 to 5; and * denotes a binding site to a part of the curable compound other than the group represented by formula (1), provided that no two

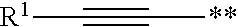

groups are in an ortho position to each other on Ar¹, wherein ** denotes the point of attachment to an aromatic ring carbon of Ar¹; and a solvent.

In accordance with a further aspect of the invention, coated substrates are provided. The coated substrates comprise: an electronic device substrate; a photoresist underlayer formed from a photoresist underlayer composition as described herein on a surface of the electronic device substrate; and a photoresist composition layer over the photoresist underlayer. The layer may function, in addition to its use as a photoresist underlayer, as a planarizing layer, a gap-filling layer, a protective layer, or a combination thereof.

In accordance with a further aspect of the invention, methods of forming electronic devices are provided. The methods comprising: (a) providing an electronic device substrate; (b) coating a layer of a photoresist underlayer composition as described herein on a surface of the electronic device substrate; and (c) curing the layer of the photoresist underlayer composition to form a photoresist underlayer. In a further aspect, the method further comprises: (d) forming a photoresist layer over the photoresist underlayer; (e) patternwise exposing the photoresist layer to activating radiation; (f) developing the exposed photoresist layer to form a pattern in the photoresist layer; and (g) transferring the pattern to the photoresist underlayer. In a further aspect, the method further comprises coating one or more of a silicon-containing layer, an organic antireflective coating layer, or a combination thereof over the photoresist underlayer before step (d). In a further aspect, the method further comprises transferring the pattern to the one or more of the silicon-containing layer, the organic antireflective coating layer, or the combination thereof after step (f) and before step (g). In a further aspect, the method further comprises: (h) transferring the pattern to a layer of the electronic device substrate below the patterned photoresist underlayer; and (i) removing the patterned photoresist underlayer.

It will be understood that when an element is referred to as being “on” or “over” another element, it can be directly adjacent to the other element or intervening elements may be present therebetween. In contrast, when an element is referred to as being “directly on” another element, there are no intervening elements present. As used herein, the term “and/or” includes any and all combinations of one or more of the associated listed items.

It will also be understood that although the terms first, second, third, etc. may be used herein to describe various elements, components, regions, layers and/or sections, these elements, components, regions, layers and/or sections should not be limited by these terms. These terms are only used to distinguish one element, component, region, layer or section from another element, component, region, layer or section. Thus, a first element, component, region, layer or section discussed below could be termed a second element, component, region, layer or section without departing from the teachings of the present invention.

As used throughout this specification, the following abbreviations shall have the following meanings, unless the context clearly indicates otherwise: ° C.=degree Celsius; g=gram; mg=milligram; L=liter; mL=milliliter; Å=angstrom; nm=nanometer; μm=micron=micrometer; mm=millimeter; sec.=second; min.=minute; hr.=hour; DI=deionized; and Da=dalton. “wt %” refers to percent by weight based on the total weight of a referenced composition, unless otherwise specified.

Unless otherwise noted, “aliphatic”, “aromatic”, “alkyl”, and “aryl” include heteroaliphatic, heteroaromatic, heteroalkyl and heteroaryl, respectively. The terms “heteroaliphatic”, “heteroaromatic”, “heteroalkyl”, “heteroaryl”, and the like, refer to aliphatic, aromatic, alkyl, and aryl, respectively, with one or more heteroatoms, such as nitrogen, oxygen, sulfur, phosphorus, or silicon, replacing one or more carbon atoms within the radical, for example, as in an ether or a thioether.

“Aliphatic” refers to open chain (linear, or branched) and cyclic aliphatic unless otherwise specified. Aliphatic structures may be saturated (e.g., alkanes) or unsaturated (e.g., alkenes or alkynes). Aliphatic refers to an aliphatic radical, and includes aliphatic monoradicals, diradicals, and higher-radicals. Unless otherwise noted. “Aliphatic” includes “heteroaliphatic”. In a preferred aspect, the aliphatic radical does not include hetero atoms.

“Alkyl” refers to linear, branched and cyclic alkyl unless otherwise specified. As used herein, “alkyl” refers to an alkane radical, and includes alkane monoradicals, diradicals (alkylene), and higher-radicals. Unless otherwise noted, “alkyl” includes “heteroalkyl”. In a preferred aspect, the alkyl radical does not include heteroatoms. If no number of carbons is indicated for any alkyl or heteroalkyl, then 1-12 carbons are contemplated.

“Aromatic” and “Aryl” include aromatic carbocycles and aromatic heterocycles. The terms “aromatic” and “aryl” refer to an aromatic radical, and includes monoradicals, diradicals (arylene), and higher-radicals. In a preferred aspect, the aromatic or aryl radical is an aromatic carbocycle.

Unless otherwise indicated, “substituted” refers to a moiety having one or more of its hydrogens replaced with one or more non-hydrogen substituents chosen from halogen, C₁₋₆ alkyl, halo-C₁₋₆ alkyl, C₁₋₆ alkoxy, halo-C₁₋₆ alkoxy, C₅₋₃₀ aryl, and C₅₋₃₀ aryloxy, preferably from halogen, C₁₋₆ alkyl, halo-C₁₋₄ alkyl, C₁₋₆ alkoxy, halo-C₁₋₄ alkoxy, and phenyl, and more preferably from halogen, C₁₋₆ alkyl, C₁₋₆ alkoxy, phenyl, and phenoxy. Unless otherwise specified, a substituted moiety preferably has from 1 to 3 substituents, and more preferably 1 or 2 substituents. “Halo” refers to fluoro, chloro, bromo, and iodo.

“Oligomer” and “oligomeric” refer to a low molecular weight polymer that includes a few, for example, from 2 to 10 total units and that are capable of further curing. As used herein, the term “polymer” includes oligomers. By the term “curing” is meant any process, such as polymerization or condensation, that increases the overall molecular weight of the coated underlayer materials, removes solubility enhancing groups from the present oligomers, or both increases the overall molecular weight and removes solubility enhancing groups. “Curable” refers to any material capable of being cured under certain conditions. As used herein, “gap” refers to any aperture on a semiconductor substrate that is intended to be filled with a gap-filling composition.

The articles “a” and “an” refer to the singular and the plural. Unless otherwise noted, all amounts are percent by weight and all ratios are by weight. All numerical ranges are inclusive and combinable in any order, except where it is clear that such numerical ranges are constrained to add up to 100%.

### DETAILED DESCRIPTION OF THE INVENTION

Photoresist underlayer compositions of the invention comprise a curable compound and a solvent. The curable compound comprises a group of the following formula (1):

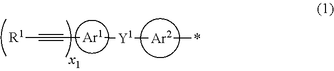

wherein: R¹ is each independently H, C₁₋₃₀ alkyl, or C₃₋₃₀ cycloalkyl; Ar¹ is an aromatic ring or a fused aromatic ring system having from 5 to 30 carbon atoms, wherein Ar¹ is substituted or unsubstituted; Ar² is an aromatic ring chosen from a 6-membered carbocyclic aromatic ring, a 5- or 6-membered heteroaromatic ring, or a fused aromatic ring system having from 5 to 30 carbon atoms, wherein Ar² optionally comprises a fused cyclic imide moiety, a fused oxazole moiety, a fused imidazole moiety, or a fused thiazole moiety, and wherein Ar² is substituted or unsubstituted; Y¹ is a single covalent bond, or is selected from —O—, —C(O)—, —C(O)O—, —S—, —S(O)₂—, —N(R²)—, —C(O)N(R²)—, —C(O)N(R²)C(O)—, —(CH²)_{y}—, or a combination thereof, wherein R² is H, C₁₋₁₀ alkyl, C₂₋₁₀ unsaturated hydrocarbyl, C₅₋₃₀ aryl, C(O)R³, or S(O)₂R³, wherein R³ is chosen from H, C₁₋₁₀ alkyl, C₂₋₁₀ unsaturated hydrocarbyl, and C₅₋₃₀ aryl, and y is an integer from 1 to 6; x₁ is an integer from 2 to 5; and * denotes a binding site to a part of the curable compound other than the group represented by formula (1), provided that no two

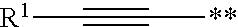

groups are in an ortho position to each other on Ar¹, wherein ** denotes the point of attachment to an aromatic ring carbon of Ar¹. The

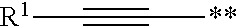

groups are also referred to herein as “R¹ alkynyl group” or “R¹ alkynyl groups.”

It is preferred that each R¹ is independently chosen from H, C₁₋₆ alkyl, or C₃₋₁₄ cycloalkyl, and more preferably each R¹ is H. It is preferred that each Ar¹ is independently chosen from pyridine, benzene, naphthalene, acenaphthylene, quinoline, isoquinoline, fluorene, carbazole, anthracene, phenanthrene, pyrene, coronene, triphenylene, chrysene, phenalene, benz[a ]anthracene, dibenz[a,h ]anthracene, benzo[a]pyrene, or pentacene, and more preferably from benzene, naphthalene, anthracene, phenanthrene, pyrene, coronene, triphenylene, chrysene, phenalene, benz[a]anthracene, dibenz[a,h]anthracene, and benzo[a]pyrene, and even more preferably from benzene, naphthalene, anthracene, phenanthrene, pyrene, coronene, triphenylene, chrysene, and phenalene, and most preferably each Ar¹ is benzene or naphthalene.

It is preferred that Ar² is chosen from pyridine, benzene, naphthalene, acenaphthylene, quinoline, isoquinoline, fluorene, carbazole, anthracene, phenanthrene, pyrene, coronene, triphenylene, chrysene, phenalene, benz[a]anthracene, dibenz[a,h]anthracene, benzo[a]pyrene, or pentacene, and more preferably from benzene, naphthalene, anthracene, phenanthrene, pyrene, coronene, triphenylene, chrysene, phenalene, benz[a]anthracene, dibenz[a,h]anthracene, and benzo[a]pyrene, and even more preferably from benzene, naphthalene, anthracene, phenanthrene, pyrene, coronene, triphenylene, chrysene, and phenalene, and most preferably each Ar² is benzene or naphthalene. Optional substituents for Ar¹ and Ar² include, for example, halogen, C₁₋₆ alkyl, C₁₋₆ haloalkyl, C₁₋₆ alkoxy, C₁₋₆ haloalkoxy, phenyl, and phenoxy.

Ar² is preferably C₆₋₅₀ carbocyclic aromatic or C₂₋₅₀ heterocyclic aromatic having a single or fused ring system. Suitable aromatic cores include, for example, those chosen from pyridine, benzene, naphthalene, quinoline, isoquinoline, carbazole, anthracene, phenanthrene, pyrene, coronene, triphenylene, chrysene, phenalene, benz[a]anthracene, dibenz[ a,h]anthracene, oxazole, isooxazole, thiazole, isothiazole, triazole and benzo[a]pyrene, more preferably from benzene, naphthalene, carbazole, anthracene, phenanthrene, pyrene, coronene, triphenylene, chrysene, phenalene, benz[a]anthracene, dibenz[a,h]anthracene, and benzo[a]pyrene, and still more preferably from benzene, naphthalene, anthracene, phenanthrene, pyrene, coronene, triphenylene, chrysene, and phenalene. The aliphatic cores may be substituted or unsubstituted, straight-chained, branched-chained or cyclic, and saturated or unsaturated (alkanes, alkenes or alkynes). Suitable aliphatic and heteroaliphatic cores include, for example, those chosen from methane, ethane, propane, butane, pentane, hexane, heptane, octane, nonane, decane, methylmethane, dimethylmethane, dimethylether, butene, butyne, dimethyl sulfide, trimethylamine, and tetramethylsilane, cyclopropane, cyclobutane, cyclopentane, cyclohexane, cycloheptane, cyclooctane, cyclononane, cyclodecane, cyclopropene, cyclobutene, cyclopentene, cyclohexene, cycloheptene, 1,3-cyclohexadiene, 1,4-cyclohexadiene, 1,5-cyclooctadiene, norbornene, adamantane, tetrahydropyran, tetrahydrothiophene, pyrrolidine, tetrahydro-2H-pyran, tetrahydro-2H-thiopyran, piperidine, and dioxane. It is typical that x₁ is an integer from 2 to 4; and more typically x₁=1.

It is typical that two of the R¹ alkynyl groups are on the same aromatic ring of Ar¹, with such two groups typically being present in a meta position with respect to reach other. The curable compounds may include one or more groups of formula (1). For example, the curable compounds may include from 1 to 10 groups of formula (1), more typically from 1 to 6, from 1 to 4, or from 2 to 4 groups of formula (1). Where the curable compounds include a plurality of groups of formula (1), each R¹, Ar¹, Ar², Y¹, and x¹ may be independently chosen.

In a preferred aspect, the curable compound is of the following formula (2):

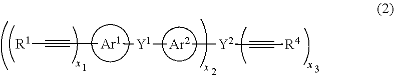

wherein: each R¹, Ar¹, Ar², Y¹, and x₁ are independently selected and are as described above with respect to formula (1); R⁴ is each independently H, C₁₋₃₀ alkyl, or C₃₋₃₀ cycloalkyl; x₂ is an integer from 1 to 10; x₃ is an integer from 0 to 5; and Y² is a single covalent bond, or a group having a valency of x₂+x₃. Y² is not particularly limited. When x₂+x₃=1, Y₂ is a monovalent atom or monovalent group, and when x₂+x₃=2 or more, Y₂ is a single covalent bond or a linking group.

In one preferred embodiment, Y₂ is a single covalent bond. In another preferred embodiment, Y₂ is a divalent or trivalent linking group. Exemplary linking groups for Y₂ include, but are not limited to, one or more of O, S, N(R⁵)ᵣ, S(O), S(O)₂, C(O), C(O)O, C(O)N(R⁵), C(O)N(R⁵)C(O), CR⁶R⁷, a bis-imide moiety, a bis-etherimide moiety, a bis-ketoimide moiety, a bis-benzoxazole moiety, a bis-benzimidazole moiety, a bis-benzothiazole moiety, C₁₋₃₀ alkylene, C₃₋₃₀ cycloalkylene, C₃₋₃₀ heterocycloalkylene, C₆₋₃₀ arylene, C₃₋₃₀ heteroarylene, and combinations thereof, wherein: R⁵ is hydrogen, C₁₋₃₀ alkyl, C₁₋₃₀ heteroalkyl, C₃₋₃₀ cycloalkyl, C₁₋₃₀ heterocycloalkyl, C₂₋₃₀ alkenyl, C₂₋₃₀ alkynyl, C₆₋₃₀ aryl, C₇₋₃₀ arylalkyl, C₇₋₃₀ alkylaryl, C₂₋₃₀ heteroaryl, C₃₋₃₀ heteroarylalkyl, C₃₋₃₀ alkylheteroaryl, **—C(═O)—C₅₋₃₀-aryl, or **—S(═O)₂—C₅₋₃₀-aryl, wherein ** is the point of attachment to N; r is 0 or 1; R⁶ and R⁷ are independently chosen from H, C₁₋₁₀ alkyl and C₅₋₁₀ aryl, and R⁶ and R⁷ may be taken together along with the carbon to which they are attached to form a 5- or 6-membered ring which may be fused to one or more aromatic rings.

A suitable linking group when Y²═CR⁶R⁷ is a fluorenyl moiety of the following formula (A)

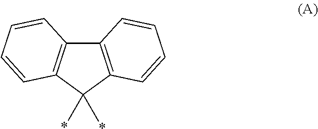 [SMILES: *C1(*)c2ccccc2-c2ccccc21]

wherein * each denotes the point of attachment such as to different Ar² groups. A suitable bis-imide moiety linking group for Y² is shown by formula (B) and formula (C) where Z¹ is a single covalent bond or a C₅₋₃₀-arylene, wherein * denotes the point of attachment such as to different Ar² groups. Suitable bis-etherimide and bis-ketoimide moieties are those of formula (C) wherein Z¹═O or —C(═O)—, respectively, and wherein * denotes the point of attachment such as to different Ar² groups. Suitable bis-benzoxazole, bis-benzimidazole, and bis-benzothiazole moieties are those of formula (D), wherein G=O, NH, and S, respectively, and wherein Z² is a single covalent bond or a C₅₋₃₀-arylene, and wherein * denotes the point of attachment such as to different Ar² groups.

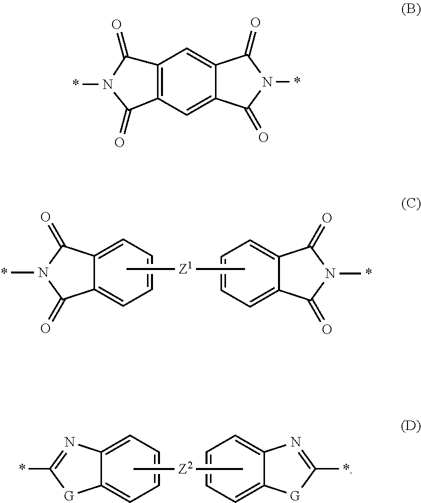

The curable compound typically has a weight average molecular weight (M_{w}) from 400 to 10,000 Dalton (Da), preferably from 400 to 3000 Da, and more preferably from 800 to 1500 Da. Molecular weights are determined by gel permeation chromatography (GPC) using polystyrene standards.

Suitable curable compounds for use in the photoresist underlayer compositions include, for example, the following:

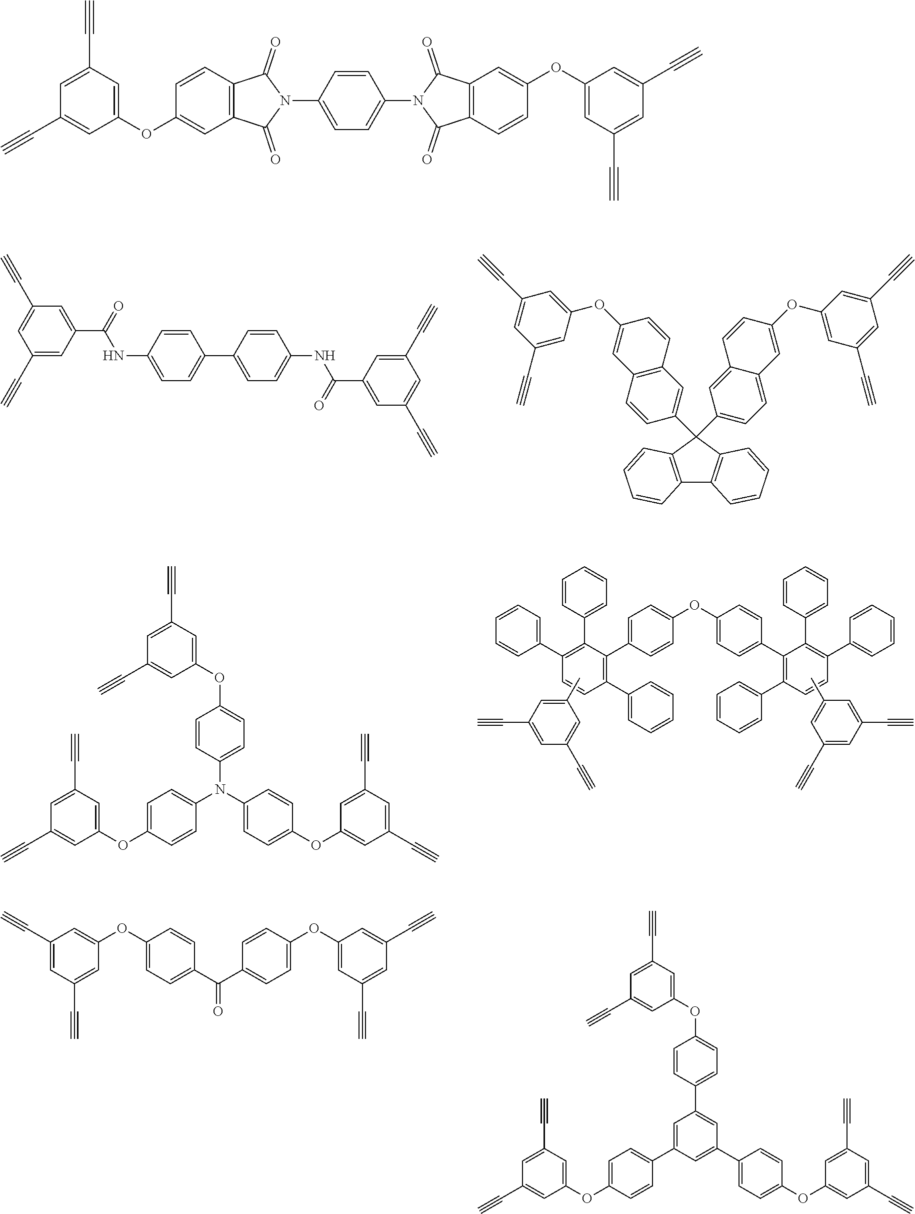

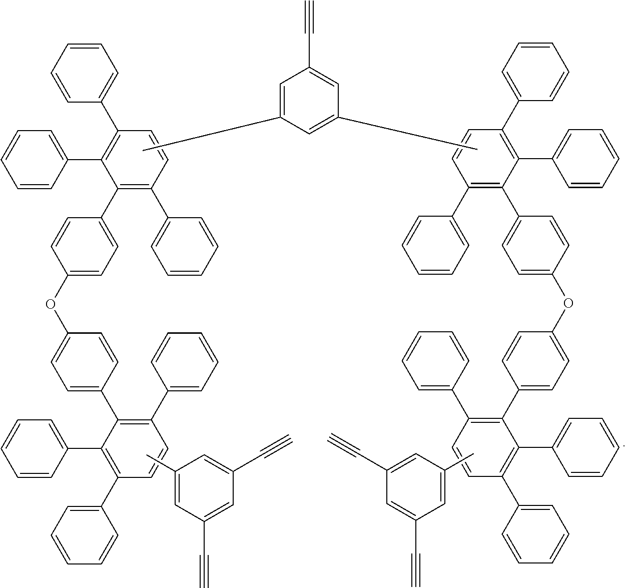

The curable compound may be present in the coating compositions in a broad range, for example, from 1 to 100 wt %, more typically from 10 to 100 wt %, from 50 to 100 wt %, from 90 to 99 wt%, or from 95 to 99 wt %, based on total solids of the coating composition. It may be desired that the curable compound is present in a relatively minor amount with respect to total solids of the composition, for example, from 1 to 50 wt % or from 1 to 30 wt %.

Curable compounds as described above can readily be made by persons skilled in the art using known synthesis techniques. For example, the compounds may be prepared by mixing reactants, for example, arylhalides or alkylhalides with aromatic alkynes, with suitable catalyst(s) such as a copper and palladium catalysts, base and solvent. The solvent is typically an organic solvent including, for example, toluene, benzene, tetrahydrofuran, dioxane, and combinations thereof. Reaction is carried at a temperature and time effective to cause reaction of the reactants in the reaction mixture to form the curable compounds. The reaction temperature is typically from 0 to 200° C. preferably from 25 to 100° C. The reaction time is typically from 5 minutes to 96 hours, preferably from 2 to 24 hours. The product compounds can be purified by techniques known in the art such as column chromatography.

The underlayer compositions comprise one or more solvents for dissolving the components of the composition and facilitating its coating on a substrate. Preferably, the one or more solvents are chosen from organic solvents conventionally used in the manufacture of electronic devices. Suitable organic solvents include, but are not limited to: hydrocarbons such as xylene, mesitylene, cumene, and limonene; ketones such as cyclopentanone, cyclohexanone (CHO), methyl ethyl ketone, and methyl-2-n-amylketone; alcohols such as 3-methoxybutanol, 3-methyl-3-methoxybutanol, 1-methoxy-2-propanol, and 1-ethoxy-2-propanol; ethers such as propylene glycol methyl ether (PGME), propylene glycol ethyl ether (PGEE), ethylene glycol monomethyl ether, propylene glycol monoethyl ether, ethylene glycol monoethyl ether, propylene glycol dimethyl ether, diethylene glycol dimethyl ether, anisole, and ethoxybenzene; esters such as propylene glycol monomethyl ether acetate (PGMEA), propylene glycol monoethyl ether acetate, ethyl lactate (EL), methyl hydroxyisobutyrate (HBM), ethyl pyruvate, butyl acetate, methyl 3-methoxypropionate, ethyl 3-ethoxypropionate, tert-butyl acetate, tert-butyl propionate, propylene glycol mono-tert-butyl ether acetate, benzyl propionate, cyclic or non-cyclic carbonate esters such as propylene carbonate, dimethyl carbonate, ethylene carbonate, and diphenyl carbonate; lactones such as gamma-butyrolactone (GBL); lactams such as N-methyl pyrrolidone; and any combination of the foregoing. Of these, preferred solvents are PGME, PGEE, PGMEA, EL, HBM, CHO, GBL, and combinations thereof. The total solvent content (i.e., cumulative solvent content for all solvents) in the underlayer compositions is from 50 to 99 wt %, typically from 80 to 99 wt %, and more typically from 90 to 99 wt %, based on the underlayer composition. The desired solvent content will depend, for example, on the desired thickness of the coated underlayer and coating conditions.

The present photoresist underlayer compositions may also comprise one or more coating additives that are typically used in such compositions, such as curing agents, crosslinking agents, surface leveling agents, flow additives, and the like. If used in the compositions, curing agents are typically present in an amount of from 1 to 20 wt %, and preferably from 1 to 3 wt % based on total solids. Crosslinking agents, if used, are typically present in an amount of from 1 to 30 wt %, and preferably from 3 to 10 wt %, based on total solids. Surface leveling agents, if used, are typically present in an amount of from 0.01 to 5 wt %, and preferably from 0.01 to 1 wt %, based on total solids. Flow additives, if used, are typically present in an amount of from 0.01 to 5 wt %, and preferably from 0.01 to 3 wt %, based on total solids.

Curing agents may optionally be used in the photoresist underlayer compositions to aid in the curing of the composition after coating. A curing agent is any component which causes curing of the composition on the surface of the substrate. Preferred curing agents are acids and thermal acid generators. Suitable acids include, but are not limited to: arylsulfonic acids such as p-toluenesulfonic acid; alkyl sulfonic acids such as methanesulfonic acid, ethanesulfonic acid, and propanesulfonic acid; perfluoroalkylsulfonic acids such as trifluoromethanesulfonic acid; and perfluoroarylsulfonic acids. A thermal acid generator is any compound which liberates acid upon exposure to heat. Thermal acid generators are well-known in the art and are generally commercially available, such as from King Industries, Norwalk, Connecticut. Exemplary thermal acid generators include, without limitation, amine blocked strong acids, such as amine blocked dodecylbenzenesulfonic acid. It will also be appreciated by those skilled in the art that certain photoacid generators are able to liberate acid upon heating and may function as thermal acid generators.

Any suitable crosslinking agent may be used in the present compositions, provided that such crosslinking agent has at least 2, and preferably at least 3, moieties capable of reacting with the present aromatic resin reaction products under suitable conditions, such as under acidic conditions. Exemplary crosslinking agents include, but are not limited to, novolac resins, epoxy-containing compounds, melamine compounds, guanamine compounds, isocyanate-containing compounds, benzocyclobutenes, and the like, and preferably any of the foregoing having 2 or more, preferably 3 or more, and more preferably 4, substituents selected from epoxy, methylol, C₁-C₁₀alkoxymethyl, and C₂-C₁₀acyloxymethyl. Suitable crosslinking agents are well-known in the art and are commercially available from a variety of sources. Examples of suitable crosslinking agents include those of formulas (3), (4), (5) and (6):

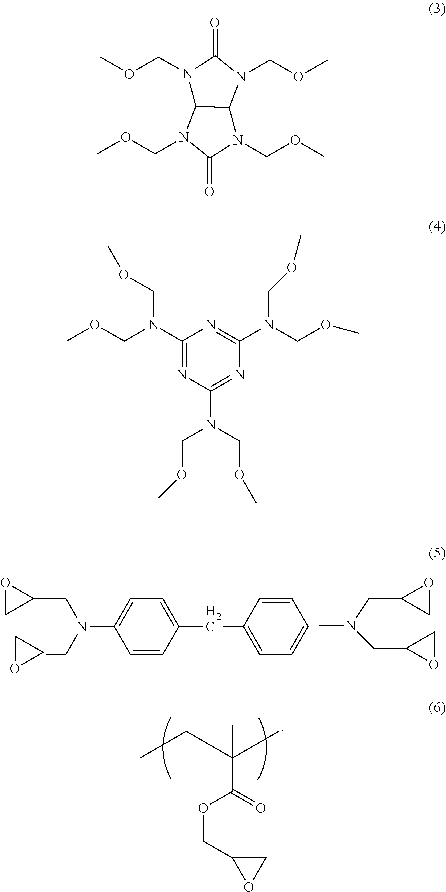

The present underlayer compositions may optionally include one or more surface leveling agents (or surfactants). While any suitable surfactant may be used, such surfactants are typically non-ionic. Exemplary non-ionic surfactants are those containing an alkyleneoxy linkage, such as ethyleneoxy, propyleneoxy, or a combination of ethyleneoxy and propyleneoxy linkages.

The underlayer compositions of the invention and films formed therefrom can exhibit beneficial characteristics in one or more of gap-filling, surface planarization, thermal stability, solvent strip-resistance and film quality properties. The present compositions preferably substantially fill and more preferably fully fill a plurality of gaps in a semiconductor substrate. Preferably, the gaps are substantially or completely void-free.

Photoresist underlayers made from the compositions described herein may be used in the formation of electronic devices according to a method comprising: (a) providing an electronic device substrate; (b) coating a layer of a photoresist underlayer composition as described herein on a surface of the electronic device substrate; and (c) curing the layer of the photoresist underlayer composition to form a photoresist underlayer.

Suitable substrates on which the underlayer compositions can be coated include electronic device substrates. A wide variety of electronic device substrates may be used in the present invention, such as: packaging substrates such as multichip modules; flat panel display substrates; integrated circuit substrates; substrates for light emitting diodes (LEDs) including organic light emitting diodes (OLEDs); semiconductor wafers; polycrystalline silicon substrates; and the like, with semiconductor wafers being preferred. Such substrates are typically composed of one or more of silicon, polysilicon, silicon oxide, silicon nitride, silicon oxynitride, silicon germanium, gallium arsenide, aluminum, sapphire, tungsten, titanium, titanium-tungsten, nickel, copper, and gold. Suitable substrates may be in the form of wafers such as those used in the manufacture of integrated circuits, optical sensors, flat panel displays, integrated optical circuits, and LEDs. As used herein, the term “semiconductor wafer” is intended to encompass “a semiconductor substrate,” “a semiconductor device,” and various packages for various levels of interconnection, including a single-chip wafer, multiple-chip wafer, packages for various levels, or other assemblies requiring solder connections. Such substrates may be any suitable size. Preferred wafer substrate diameters are 200 mm to 300 mm, although wafers having smaller and larger diameters may be suitably employed according to the present invention. As used herein, the term “semiconductor substrate” includes any substrate having one or more layers or structures which may optionally include active or operable portions of semiconductor devices. A semiconductor device refers to a semiconductor substrate upon which at least one microelectronic device has been or is being fabricated.

Optionally, a layer of an adhesion promoter may be applied to the substrate surface prior to coating of the present underlayer compositions. If an adhesion promoter is desired, any suitable adhesion promoter for polymer films may be used, such as silanes, preferably organosilanes such as trimethoxyvinylsilane, triethoxyvinylsilane, hexamethyldisilazane, or an aminosilane coupler such as gamma-aminopropyltriethoxysilane. Particularly suitable adhesion promoters include those sold under the AP 3000, AP 8000, and AP 9000S designations, available from DuPont Electronics & Industrial (Marlborough, Massachusetts).

The present underlayer compositions may be coated on the electronic device substrate by any suitable means, such as spin-coating, slot-die coating, doctor blading, curtain coating, roller coating, spray coating, dip coating, and the like. Of these, spin-coating is preferred. In a typical spin-coating method, the present compositions are applied to a substrate which is spinning at a rate of 500 to 4000 rpm for a period of 15 to 90 seconds to obtain a desired layer of the underlayer composition on the electronic device substrate. It will be appreciated by those skilled in the art that the height of the underlayer composition layer may be adjusted by changing the spin speed.

After being coated on the substrate, the underlayer composition layer is optionally baked at a relatively low temperature to remove any organic solvent and other relatively volatile components from the layer. Typically, the substrate is baked at a temperature of 80 to 150° C., although other suitable temperatures may be used. The baking time is typically from 10 seconds to 10 minutes, and preferably from 30 seconds to 5 minutes, although longer or shorter times may be used. When the substrate is a wafer, such baking step may be performed by heating the wafer on a hot plate. Following solvent removal, a layer, film or coating of the underlayer coating composition on the substrate surface is obtained.

The layer is then sufficiently cured to form an aromatic photoresist underlayer at conditions such that the film does not intermix with a subsequently applied coating layer, for example, a photoresist or other layer coated directly on the aromatic underlayer, while still maintaining the desired antireflective properties (n and k values) and etch selectivity of the underlayer film. The underlayer may be cured in an oxygen-containing atmosphere, such as air, or in an inert atmosphere, such as nitrogen, and preferably in an oxygen-containing atmosphere. This curing step is preferably conducted on a hot plate-style apparatus, though oven curing may be used. Typically, such curing is performed by heating the underlayer at a curing temperature of ≥150° C., preferably ≥170° C., and more preferably ≥200° C. The curing temperature and time selected should be sufficient to cure the aromatic underlayer. A suitable temperature rage for curing the aromatic underlayer is 150 to 450° C., preferably from 170 to 350° C., and more preferably from 200 to 250° C. Such curing step may take from 10 sec. to 10 min., preferably from 1 to 3 min., and more preferably from 1 to 2 min., although other suitable times may be used.

The initial baking step may not be necessary if the curing step is conducted in such a way that rapid evolution of the solvents and curing by-products are not allowed to disrupt the underlayer film quality. For example, a ramped bake beginning at relatively low temperatures and then gradually increasing to a temperature of ≥200° C. can give acceptable results. It can be preferable in some cases to use a multi-stage curing process, for example, a two-stage process with the first stage being a lower bake temperature of less than 150° C., and the second stage being a higher bake temperature of ≥200° C. Multi-stage curing processes can facilitate uniform filling and planarization of pre-existing substrate surface topography, for example filling of trenches and vias.

After curing of the underlayer, one or more processing layers, such as photoresists, silicon-containing layers, hardmask layers, bottom antireflective coating (or BARC) layers, and the like, may be coated on the cured underlayer. For example, a photoresist may be coated, such as by spin-coating, directly on the surface of a silicon-containing layer or other middle layer which is directly on the resin underlayer, or, alternatively, the photoresist may be coated directly on the cured underlayer. A wide variety of photoresists may be suitably used, such as those used in 193 nm (ArF) lithography, such as those sold under the EPIC™ brand available from DuPont Electronics & Industrial (Marlborough, Massachusetts) and EUV lithography. Suitable photoresists may be of either positive- or negative-type. Following coating, the photoresist layer is then imaged (exposed) using patterned activating radiation, and the exposed photoresist layer is then developed using the appropriate developer to provide a patterned photoresist layer. References herein to exposing a photoresist layer to radiation that is activating indicates that the radiation is capable of forming a latent image in the photoresist layer. The photoresist layer may be exposed to the activating radiation through a patterned photomask having optically opaque and optically transparent regions, or by direct writing. The pattern is next transferred from the photoresist layer to the underlayer by an appropriate etching technique. Typically, the photoresist is also removed during such etching step. Next, the pattern is transferred to the substrate and the underlayer is removed by appropriate etching techniques known in the art, such as by plasma etching. Following patterning of the substrate, the underlayer is removed using conventional techniques. The electronic device substrate is then processed according to conventional means.

The cured underlayer may be used as the bottom layer of a multilayer resist process. In such a process, a layer of the underlayer composition is coated on a substrate and cured as described above. Next, one or more middle layers are coated on the aromatic underlayer. For example, a silicon-containing layer or a hardmask layer may be coated directly on the aromatic underlayer. Exemplary silicon-containing layers, such as a silicon-BARC, may be deposited by spin-coating on the underlayer followed by curing, or an inorganic silicon layer such as SiON or SiO may be deposited on the underlayer by chemical vapor deposition (CVD). Any suitable hardmask may be used and may be deposited on the underlayer by any suitable technique, and cured as appropriate. Optionally, an organic BARC layer may be disposed directly on the silicon-containing layer or hardmask layer, and appropriately cured. Next, a photoresist, such as those used in 193 nm lithography, is coated directly on the silicon-containing layer (in a trilayer process) or directly on the organic BARC layer (in a quadlayer process). The photoresist layer is then imaged (exposed) using patterned activating radiation, and the exposed photoresist layer is then developed using the appropriate developer to provide a patterned photoresist layer. The pattern is next transferred from the photoresist layer to the layer directly below it, by appropriate etching techniques known in the art, such as by plasma etching. This results in a patterned silicon-containing layer in a trilayer process and a patterned organic BARC layer in a quadlayer process. If a quadlayer process is used, the pattern is next transferred from the organic BARC layer to the silicon-containing layer or hardmask layer using appropriate pattern transfer techniques, such as plasma etching. After the silicon-containing layer or hardmask layer is patterned, the aromatic underlayer is then patterned using appropriate etching techniques, such as O₂ or CF₄ plasma. Any remaining patterned photoresist and organic BARC layers are removed during etching of the aromatic underlayer. Next, the pattern is transferred to the substrate, such as by appropriate etching techniques, which also removes any remaining silicon-containing layer or hardmask layer, followed by removal of any remaining patterned aromatic underlayer to provide a patterned substrate.

The cured underlayer of the present invention may also be used in a self-aligned double patterning process. In such a process, a layer of the present underlayer composition is coated on a substrate, preferably by spin-coating. Any remaining organic solvent is removed and the underlayer composition layer is cured to form a cured underlayer. A suitable middle layer, such as a silicon-containing layer is coated on the cured underlayer. A layer of a suitable photoresist is then coated on the middle layer, such as by spin-coating. The photoresist layer is then patternwise imaged (exposed) with activating radiation, and the exposed photoresist layer is then developed using the appropriate developer to provide a patterned photoresist layer. The pattern is next transferred from the photoresist layer to the middle layer and the cured underlayer by appropriate etching techniques to expose portions of the substrate. Typically, the photoresist is also removed during such etching step. Next, a conformal silicon-containing layer is disposed over the patterned cured underlayer and exposed portions of the substrate. Such silicon-containing layer is typically an inorganic silicon layer such as SiON or SiO₂ which is conventionally deposited by CVD. Such conformal coatings result in a silicon-containing layer on the exposed portions of the substrate surface as well as over the underlayer pattern. That is, such silicon-containing layer substantially covers the sides and top of the patterned underlayer. Next, the silicon-containing layer is partially etched (trimmed) to expose a top surface of the patterned polyarylene resin underlayer and a portion of the substrate. Following this partial etching step, the pattern on the substrate comprises a plurality of features, each feature comprising a line or post of the cured underlayer with the silicon-containing layer directly adjacent to the sides of each cured underlayer feature, also referred to as a sidewall spacer. Next, the cured underlayer is removed, such as by etching, to expose the substrate surface that was under the cured underlayer pattern, and providing a patterned silicon-containing layer on the substrate surface, where such patterned silicon-containing layer is doubled (that is, twice as many lines and/or posts) as compared to the patterned cured underlayer.

In addition to their use in forming photoresist underlayers and patterns as described above, the underlayer compositions of the invention are useful in forming planarizing layers, gap-filling layers, and protective layers in the manufacture of integrated circuits. When used as such layers, one or more intervening material layers, such as silicon-containing layers, other aromatic resin layers, hardmask layers, or the like, are typically present between the cured layer of the present underlayer composition and any photoresist layer. Typically, such planarizing layers, gap-filling layers, and protective layers are ultimately patterned.

The following non-limiting examples are illustrative of the invention.

### EXAMPLES

### Synthesis Examples

### Example 1

Bis(4-(3,5-dibromophenoxy)phenyl)methanone (10.91 g), cuprous iodide (0.46 g) and triethylamine (7.29 g) were added to 80 g of 1,4-dioxane at room temperature. The reaction mixture was purged with nitrogen for 1 hour. Bis(triphenylphosphine)palladium(II) chloride (1.12 g) was added to the reaction mixture, and the mixture was heated to 70° C. Ethynyltrimethylsilane (11.53 g) was slowly added to the reaction mixture with an addition funnel. The reaction mixture was then stirred overnight at 70° C. under nitrogen. After reaction completion, the reaction mixture was cooled to room temperature, filtered and solvents were evaporated. The obtained residue was then dissolved in THF (100 g). Potassium carbonate (11.40 g), methanol (100 g) and water (10 g) were added, and the reaction mixture was stirred at room temperature overnight. Solvent was removed and water (100 mL) was added. The mixture was extracted three times with ethyl acetate, and the combined organic phase was washed with water, washed with brine, and then dried over Na₂SO₄. The solvent was removed under vacuum, and the residue was purified by column chromatography to give bis(4-(3,5-diethynylphenoxy)phenyl)methanone (Compound I-1) as a white solid (5.99 g, 81% yield).

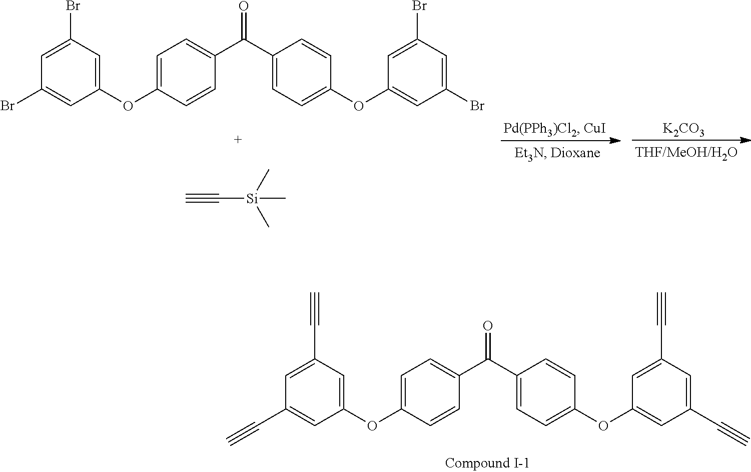

### Example 2

5,5′-((Sulfonylbis(4,1-phenylene))bis(oxy))bis(1,3-dibromobenzene) (11.49 g), cuprous iodide (0.46 g) and triethylamine (7.29 g) were added to 80 g of 1,4-dioxane at room temperature. The reaction mixture was purged with nitrogen for 1 hour. Bis(triphenylphosphine)palladium(II) chloride (1.12 g) was added to the reaction mixture, and the mixture was heated to 70° C. Ethynyltrimethylsilane (11.53 g) was slowly added to the reaction mixture with an addition funnel. The reaction mixture was then stirred overnight at 70° C. under nitrogen. After reaction completion, the reaction mixture was cooled to room temperature, filtered and solvents were evaporated. The obtained residue was then dissolved in THF (100 g). Potassium carbonate (11.40 g), methanol (100 g) and water (10 g) were added, and the reaction mixture was stirred at room temperature overnight. Solvent was removed and water (100 mL) was added. The mixture was extracted three times with ethyl acetate, and the combined organic phase was washed with water, washed with brine, and then dried over Na₂SO₄. The solvent was removed under vacuum, and the residue was purified by column chromatography to give 5,5′-((sulfonylbis(4,1-phenylene))bis(oxy))bis(1,3-diethynylbenzene) (Compound I-2) as a white solid (6.06 g, 76% yield).

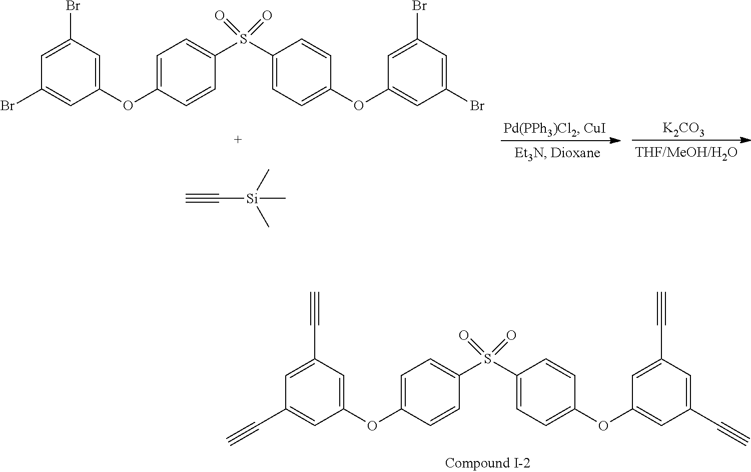

### Example 3

9,9-Bis(4-(3,5-dibromophenoxy)phenyl)-9H-fluorene (13.09 g), cuprous iodide (0.46 g) and triethylamine (7.29 g) were added to 80 g of 1,4-dioxane at room temperature. The reaction mixture was purged with nitrogen for 1 hour. Bis(triphenylphosphine)palladium(II) chloride (1.12 g) was added to the reaction mixture, and the mixture was heated to 70° C. Ethynyltrimethylsilane (11.53 g) was slowly added to the reaction mixture with an addition funnel. The reaction mixture was then stirred overnight at 70° C. under nitrogen. After reaction completion, the reaction mixture was cooled to room temperature, filtered and solvents were evaporated. The obtained residue was then dissolved in THF (100 g). Potassium carbonate (11.40 g), methanol (100 g) and water (10 g) were added, and the reaction mixture was stirred at room temperature overnight. Solvent was removed and water (100 mL) was added. The mixture was extracted three times with ethyl acetate, and the combined organic phase was washed with water, washed with brine, and then dried over Na₂SO₄. The solvent was removed under vacuum, and the residue was purified by column chromatography to give 9,9-bis(4-(3,5-diethynylphenoxy)phenyl)-9H-fluorene (Compound I-3) as a white solid (7.47 g, 78% yield).

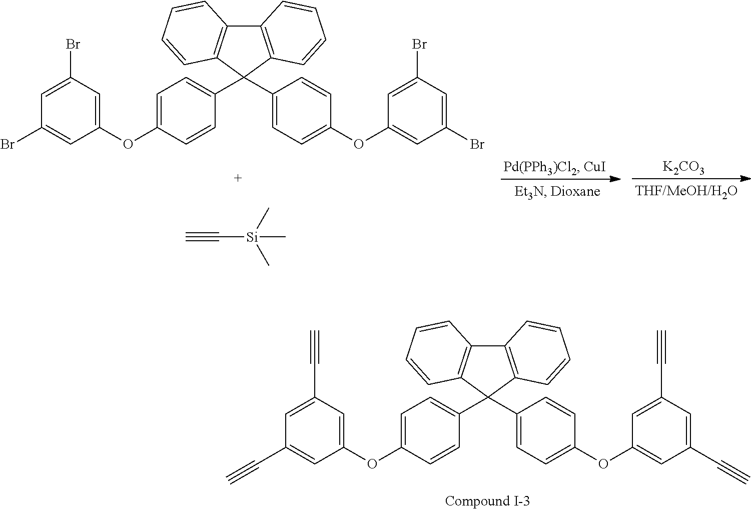

### Example 4

4,4′-(oxybis(4,1-phenylene))bis(2,3,5-triphenylcyclopenta-2,4-dien-1-one) (7.83 g), 1,3,5-triethynylbenzene (3.75 g) and gamma-butyrolactone (50.36 g) were added to a 500 mL flask. The mixture was stirred at 120° C. for 6 hours under nitrogen. After reaction completion, the reaction mixture was cooled to room temperature, precipitated into 100 ml of water, and filtered. The residue solid was purified by column chromatography to give Compound I-4 as a pale yellow solid (3.50 g, 34% yield) and Compound I-5 as a pale yellow solid (2.06 g, 20% yield).

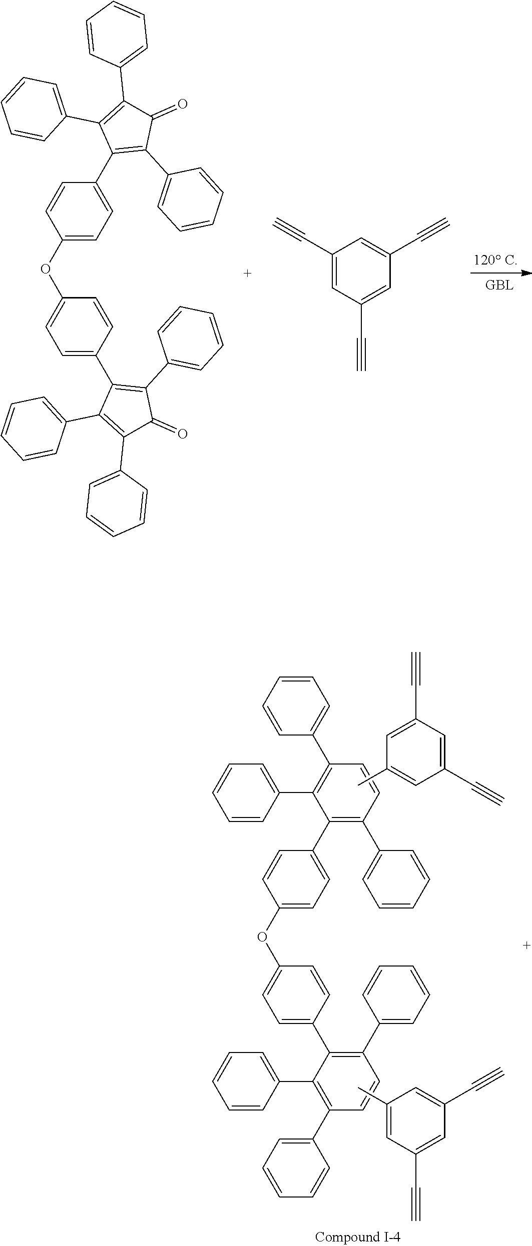

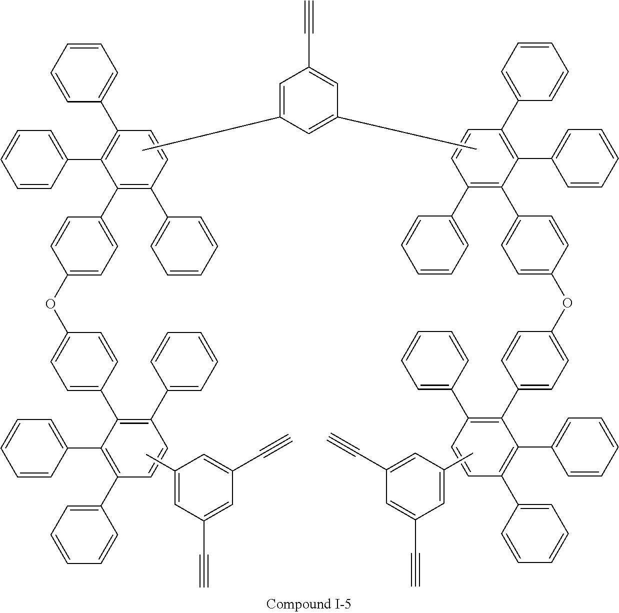

### Example 5 (Comparative)

A mixture of 4,4′-(oxybis(4,1-phenylene))bis(2,3,5-triphenylcyclopenta-2,4-dien-1-one) and 1,3,5-tris(phenylethynyl)benzene in a mole ratio of 1:1.08 and concentration of 30-40 wt % of solids in gamma-butyrolactone was heated to a target temperature of 200° C. until a Mw of 8800 Da was obtained (approximately 10-15 hours). The reactor was then cooled to 120° C. to stop further reaction. Cyclohexanone was added to dilute the polyarylene polymer to form a storage solution. 1 L of the storage solution was added to 10 L of isopropanol over 30 minutes. The solution was then stirred for 1 hour, and the resulting precipitate was filtered, washed two times with 1 L of IPA, air-dried, and vacuum-dried at 50° C. to provide 327.3 g of oligomeric Compound I-6 (Comparative).

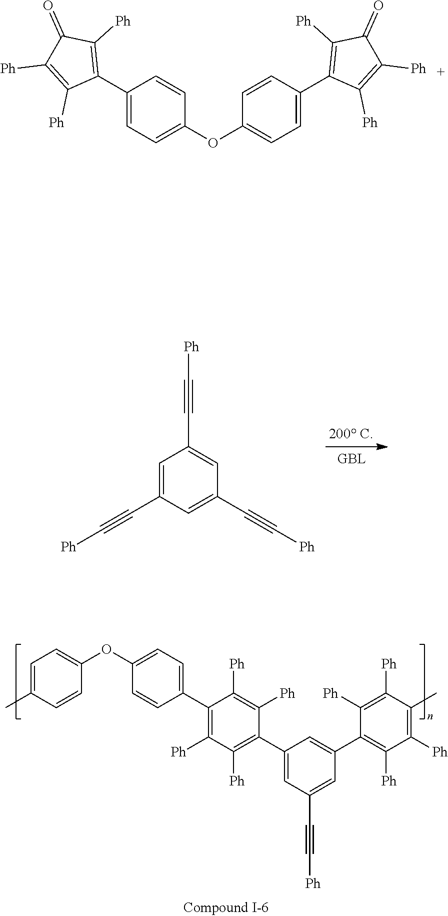

### Example 6 (Comparative)

4-Iodophenyl acetate (24.75 g), cuprous iodide (0.17 g), and triethylamine (27.32 g) were added to 22.82 g of 1,4-dioxane at room temperature. The reaction mixture was purged with nitrogen for 1 hour. Bis(triphenylphosphine)palladium(II) chloride (0.63 g) was added to the reaction mixture, and the mixture was heated to 70° C. A solution of 1,3,5-triethynylbenzene (4.5 g) in degassed 1,4-dioxane (20 g) was then slowly added to reaction mixture via syringe pump. After completion of addition, the reaction was stirred overnight at 70° C. under nitrogen. After completion of the reaction, the reaction mixture was cooled to room temperature and solvents were evaporated. The residue was diluted with ethyl acetate and filtered to remove the solid. The solution was evaporated, and the residue was purified by column chromatography to give a light yellow solid. This obtained solid was then dissolved in THF (38 g) under nitrogen. Lithium hydroxide monohydrate (3.81 g) and water (16 g) were added, and the mixture was stirred for 1 hour at 60° C. The mixture was then cooled to room temperature, and the solvent was removed. The residue was diluted with ethyl acetate and water, and then treated with hydrochloric acid until the pH of the aqueous layer was 1. The organic phase was separated and the aqueous phase was extracted with ethyl acetate. The organic layers were combined, and washed with water. The solvent was removed under vacuum, and the residue was purified by column chromatography to obtain 7.7 g (61% yield) of 1,3,5-tris((4-hydroxyphenyl)ethynyl)benzene Compound I-7 (Comparative) as a light yellow solid.

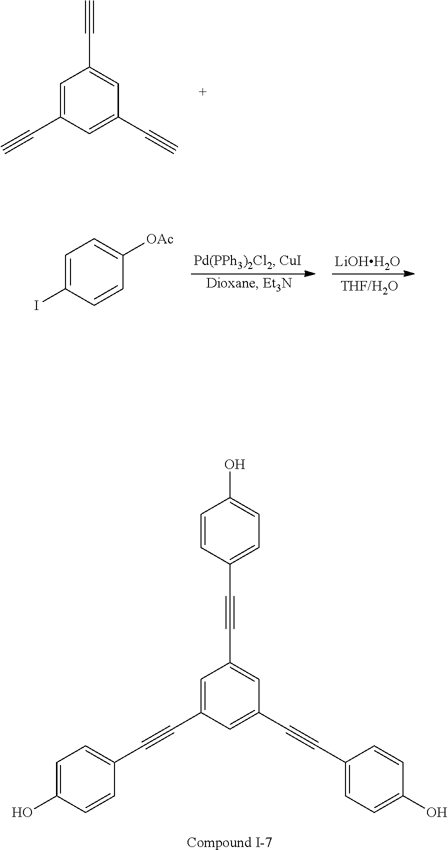 [SMILES: C#Cc1cc(C#C)cc(C#C)c1 ; CC(=O)Oc1ccc(I)cc1 ; "Dioxane, Et3N" ; "LiOH·H2O" ; Oc1ccc(C#Cc2cc(C#Cc3ccc(O)cc3)cc(C#Cc3ccc(O)cc3)c2)cc1 ; "Compound I-7"]

### Solubility Evaluation

### Examples 7-13

Curable compounds as prepared in the synthesis examples inclusive of any solvent added during the example description above were diluted with PGME or PGMEA to 5 wt % solids based on total weight of the mixture. The resulting mixtures were shaken and then visually inspected. The mixtures were also tested with a turbidity meter (Orbeco-Hellige Co). If the turbidity value was 1.00 or less, the compound was rated soluble (“S”). If the turbidity value was greater than 1.00, the compound was rated not soluble (“NS”). The results for turbidity measurement are reported in Table 1.

TABLE 1

Solubility (5 wt % solids)

| Example | Compound | PGMEA | PGME |
|---|---|---|---|
| 7 | I-1 | S | S |
| 8 | I-2 | S | S |
| 9 | I-3 | S | S |
| 10 | I-4 | S | S |
| 11 | I-5 | S | S |
| 12 (Comp.) | I-6 | S | NS |
| 13 (Comp.) | I-7 | S | S |

### Thermal Stability Evaluation

### Example 14-20

Thermal stability of curable compounds I-1 to I-7 was evaluated using a TA-Instruments Q500 Thermal Gravimetric Analyzer under the following conditions: air atmosphere, 10° C./minute temperature ramp from room temperature to 700° C. The temperature at which the material lost 5% of its weight (“Td_{5%}”) was determined. The results are set forth in Table 2.

TABLE 2

| Example | Compound | Td_{5%} (° C.) |
|---|---|---|
| 14 | I-1 | >500 |
| 15 | I-2 | >500 |
| 16 | I-3 | >500 |
| 17 | I-4 | >500 |
| 18 | I-5 | >500 |
| 19 (Comp.) | I-6 | >500 |
| 20 (Comp.) | I-7 | 470 |

### Underlayer Composition Preparation

### Examples 21-27

The materials shown in Table 3 were combined to form underlayer compositions having a solids content of about 4.5 wt %, and the dilution of each solution was adjusted for a target coated film thickness (after curing) of 100 nm.

TABLE 3

| | Underlayer | Curable | | |
|---|---|---|---|---|
| Example | Composition | Compound | Surfactant | Solvent |
| 21 | UC-1 | I-1 (99.9) | PF-656 (0.1) | PB |
| 22 | UC-2 | I-2 (99.9) | PF-656 (0.1) | PB |
| 23 | UC-3 | I-3 (99.9) | PF-656 (0.1) | PB |
| 24 | UC-4 | I-4 (99.9) | PF-656 (0.1) | PB |
| 25 | UC-5 | I-5 (99.9) | PF-656 (0.1) | PB |
| 26 (Comp.) | UC-6 | I-6 (99.9) | PF-656 (0.1) | PB |
| 27 (Comp.) | UC-7 | I-7 (99.9) | PF-656 (0.1) | PB |

PB = PGMEA/benzyl benzoate (97/3 by weight); PF-656 = PolyFox PF-656 surfactant (Synthomer); numerical values for solid components are wt % based on total solids.

### Solvent Strip Resistance Evaluation

### Examples 28-34

Underlayer compositions were spin-coated onto a respective 200 mm silicon wafer on an ACT-8 Clean Track (Tokyo Electron Co.) at 1500+/−200 rpm, and then cured at the temperature and time set forth in Table 4 to form a film. Initial film thickness was measured with a Therma-Wave OptiProbe™ metrology tool. A PGME/PGMEA (70/30 by weight) remover was then applied to each of the films for 90 seconds followed by a post-strip bake in air at 105° C. for 60 seconds. The thickness of each film was again measured to determine the amount of film thickness lost. The difference in film thickness before and after contact with the remover is set forth in Table 4 as the percentage of film thickness remaining on the wafer (% Film Remaining). This value is indicative of the degree of crosslinking of the polymeric layer.

TABLE 4

| | Underlayer | Cure | % Film |
|---|---|---|---|
| Example | Composition | Temp/Time | Remaining |
| 28 | UC-1 | 260° C./60 sec. | >99 |
| 29 | UC-2 | 260° C./60 sec. | >99 |
| 30 | UC-3 | 260° C./60 sec. | >99 |
| 31 | UC-4 | 260° C./60 sec. | >99 |
| 32 | UC-5 | 280° C./60 sec. | >99 |
| 33 (Comp.) | UC-6 | 300° C./60 sec. | <10 |
| 34 (Comp.) | UC-7 | 230° C./60 sec. | >99 |

### Gap-Fill and Planarization Evaluation

### Examples 33-36

2×2-inch silicon substrates having various patterned features were used to evaluate gap-fill and planarization performance of underlayer compositions of the invention. The features were formed in a 100 nm-thick PECVD silicon oxide layer coated on the substrates. Prior to coating the underlayer compositions, the substrates underwent a dehydration bake at 150° C. for 60 seconds. The underlayer compositions were each coated on a respective substrate with an ACT-8 Clean Track (Tokyo Electron Co.) at 1500+/−200 rpm to achieve a target film thickness after curing of approximately 100 nm. The coated compositions were cured on a hot plate at the temperature and time conditions set forth in Table 5. Cross-sectional images were taken on a Hitachi High Technologies Corp. S4800 CD-SEM.

Gap-fill performance was evaluated using the SEM images by visual inspection of 45 nm 1:1 line/space (trench) patterns overcoated with the underlayer. Gap-fill performance was deemed to be “Good” if no voids or bubbles were observed in the trench patterns, and “Poor” if any voids or bubbles were observed. Planarization performance of the underlayer compositions was evaluated from the SEM images using Hitachi offline CD measurement software by measuring the difference in underlayer thickness in dense trench patterns and open areas of the film (ΔFT). Underlayers having a ΔFT less than 30 nm were considered to have “Good” planarization and those having a ΔFT of 30 nm or more were considered to have “Poor” planarization. The results are set forth in Table 5.

TABLE 5

| | Underlayer | | | |
|---|---|---|---|---|
| Example | Composition | Cure Temp/Time | Gap-Fill | Planarization |
| 29 | UC-4 | 260° C./60 sec. | Good | Good |
| 30 | UC-5 | 280° C./60 sec. | Good | Good |
| 31 (Comp.) | UC-6 | 450° C./60 sec. | Poor | Poor |
| 32 (Comp.) | UC-7 | 230° C./60 sec. | Good | Good |

1. A photoresist underlayer composition, comprising:
a curable compound comprising a group of the following formula (1): 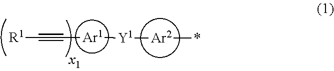
wherein: R¹ is each independently H, C₁₋₃₀ alkyl, or C₃₋₃₀ cycloalkyl; Ar¹ is an aromatic ring or a fused aromatic ring system having from 5 to 30 carbon atoms, wherein Ar¹ is substituted or unsubstituted; Ar² is an aromatic ring chosen from a 6-membered carbocyclic aromatic ring, a 5- or 6-membered heteroaromatic ring, or a fused aromatic ring system having from 5 to 30 carbon atoms, wherein Ar² optionally comprises a fused cyclic imide moiety, a fused oxazole moiety, a fused imidazole moiety, or a fused thiazole moiety, and wherein Ar² is substituted or unsubstituted; Y¹ is a single covalent bond, or is selected from —O—, —C(O)—, —C(O)O—, —S—, —S(O)₂—, —N(R₂)—, —C(O)N(R₂)—, —C(O)N(R₂)C(O)—, —(CH₂)_{y}—, or a combination thereof, wherein R² is H, C₁₋₁₀ alkyl, C₂₋₁₀ unsaturated hydrocarbyl, C₅₋₃₀ aryl, C(O)R³, or S(O)₂R³, wherein R³ is chosen from H, C₁₋₁₀ alkyl, C₂₋₁₀ unsaturated hydrocarbyl, and C₅₋₃₀ aryl, and y is an integer from 1 to 6; x₁ is an integer from 2 to 5; and * denotes a binding site to a part of the curable compound other than the group represented by formula (1), provided that no two 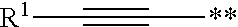
groups are in an orto position to each other on Ar¹, wherein ** denotes the point of attachment to an aromatic ring carbon of Ar¹; and
a solvent.

2. The photoresist underlayer composition of claim 1, wherein Ar¹ is pyridine, benzene, naphthalene, acenaphthylene, quinoline, isoquinoline, fluorene, carbazole, anthracene, phenanthrene, pyrene, coronene, triphenylene, chrysene, phenalene, benz[a]anthracene, dibenz[a,h]anthracene, benzo[a]pyrene, or pentacene.

3. The photoresist underlayer composition of claim 1, wherein Ar¹ is a C₆ carbocyclic aromatic ring.

4. The photoresist underlayer composition of claim 1, wherein R¹ is H.

5. The photoresist underlayer composition of claim 1, wherein the curable compound is of the following formula (2): 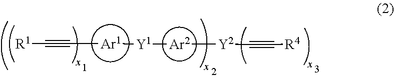
wherein: R¹ and R⁴ are each independently H, C₁₋₃₀ alkyl, or C₃₋₃₀ cycloalkyl; Ar¹ is each independently an aromatic ring or a fused aromatic ring system having from 5 to 30 carbon atoms, wherein Ar¹ is substituted or unsubstituted; Ar² is each independently an aromatic ring chosen from a 6-membered carbocyclic aromatic ring, a 5- or 6-membered heteroaromatic ring, or a fused aromatic ring system having from 5 to 30 carbon atoms, wherein Ar² optionally comprises a fused cyclic imide moiety, and wherein Ar² is substituted or unsubstituted; Y¹ is each independently a single covalent bond, or is independently selected from —O—, —C(O)—, —C(O)O—, —S—, —S(O)₂—, —N(R₂)—, —C(O)N(R²)—, —C(O)N(R²)C(O)—, —(CH²)_{y}—, or a combination thereof, wherein R² is H, C₁₋₁₀-alkyl, C₂₋₁₀-unsaturated hydrocarbyl, C₅₋₃₀-aryl, C(O)R³, or S(O)₂R³, wherein R³ is chosen from H, C₁₋₁₀-alkyl, C₂₋₁₀-unsaturated hydrocarbyl, and C₅₋₃₀-aryl, and y is an integer from 1 to 6; x₁ is an integer from 1 to 5, provided that x₁ is an integer from 2 to 5 for at least one 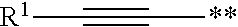
group, and no two 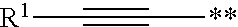
groups are in an ortho position to each other on Ar¹, wherein ** denotes the point of attachment to an aromatic ring carbon of Ar¹; x₂ is an integer from 1 to 5; x₃ is an integer from 0 to 5; and Y² is a single covalent bond, or an atom or group having a valency of x₂+x₃.

6. The photoresist underlayer composition of claim 1, comprising a first organic solvent and a second organic solvent that is different than the first organic solvent, wherein the first organic solvent has a boiling point of less than 200° C. and the second organic solvent has a boiling point of 200 ° C. or greater.

7. The photoresist underlayer composition of claim 1, further comprising a curing agent, a surface leveling agent, or a flow additive.

8. The photoresist underlayer composition of claim 1, further comprising a polymer.

9. The photoresist underlayer composition of claim 1, further comprising a crosslinker.

10. A coated substrate, comprising:
an electronic device substrate;
a photoresist underlayer formed from the photoresist underlayer composition of any one of claim 1 on a surface of the electronic device substrate; and
a photoresist composition layer over the photoresist underlayer.

11. A method of forming an electronic device, comprising:
(a) providing an electronic device substrate;
(b) coating a layer of the photoresist underlayer composition of claim 1 on a surface of the electronic device substrate; and
(c) curing the layer of the photoresist underlayer composition to form a photoresist underlayer.

12. The method of claim 11, further comprising:
(d) forming a photoresist layer over the photoresist underlayer;
(e) patternwise exposing the photoresist layer to activating radiation;
(f) developing the exposed photoresist layer to form a pattern in the photoresist layer; and
(g) transferring the pattern to the photoresist underlayer.

13. The method of claim 12, further comprising coating one or more of a silicon-containing layer, an organic antireflective coating layer, or a combination thereof over the photoresist underlayer before step (d).

14. The method of claim 13, further comprising transferring the pattern to the one or more of the silicon-containing layer, the organic antireflective coating layer, or the combination thereof after step (f) and before step (g).

15. The method of claim 1, further comprising:
(h) transferring the pattern to a layer of the electronic device substrate below the patterned photoresist underlayer; and
(i) removing the patterned photoresist underlayer.
